(19) United States
(12) Patent Application Publication
(10) Pub. No.: US 2024/0402518 A1
(43) Pub. Date: Dec. 5, 2024

(54) SMART REMOTELY CONTROLLED CONTACT LENS

(71) Applicant: Phi Biomed Inc., Seoul (KR)

(72) Inventors: Sei Kwang Hahn, Busan (KR); Geonhui Lee, Ulsan (KR); Jae-Yoon Sim, Gyeongsangbuk-do (KR); Jahyun Koo, Gyeonggi-do (KR); Dohee Keum, Busan (KR)

(21) Appl. No.: 18/745,069

(22) Filed: Jun. 17, 2024

Related U.S. Application Data
(62) Division of application No. 16/956,890, filed on Jun. 22, 2020, now abandoned.

(51) Int. Cl. G02C 7/04; G02C 11/00; G02C 11/04

(52) U.S. Cl. CPC G02C 7/047; G02C 11/04; G02C 11/10

(57) ABSTRACT

The present invention relates to a smart remotely controlled contact lens for diagnosing and treating diseases by using a micro-LED. The present invention can diagnose and treat diseases by using a micro-LED or-OLED disposed in a contact lens. Further, the present invention can treat various diseases by using signals according to light wavelengths detected through a photodetector to control drug release from a drug delivery system in the contact lens. The drug delivery system that is a small-sized ocular insert can be electrically controlled. Accordingly, drug can be released from the drug delivery system at a desired time, and thus the drug delivery system can be applied to treatment of various diseases. Further, the photodetector can detect the therapeutic effect in real time through light reflected from a treated target cell, and thus the disease progression in a patient can be easily and quickly checked.

### CROSS-REFERENCE TO RELATED APPLICATIONS

this divisional application claims priority to U.S. application Ser. No. 16/956,890, filed Jun. 22, 2020, which claims priority to PCT Application No. PCT/KR2017/015243, filed Dec. 21, 2017, which are each incorporated herein by reference in their entireties.

### TECHNICAL FIELD

The present invention relates to development of a smart remotely controlled contact lens for diagnosing and treating a disease.

### BACKGROUND ART

Research on smart wearable devices that are manufactured by making smart devices smaller or lighter to be worn on the body and enhance convenience is very actively progressing. Representative companies that research such smart wearable devices in earnest and launch innovative products are Samsung Electronics, Apple, Google, Nike and Adidas.

Google has recently attracted new attention by recently developing a smart contact lens following Google Glass 2.0. Like this, numerous global research companies are developing various electronic devices to diagnose and treat a human disease in tandem with the development of an e-health system. In addition, to treat a disease more conveniently and minimize injections and regular medication use, a diagnostic system that can easily control a drug delivery system using a smart phone was developed.

As a method of administering a drug to an eye to treat an eye disease, there is application of eye drops, intraocular injection or insertion of a drug by surgery. However, the application of eye drops has a limit to the amount of medicine, which can actually be put into eyes due to washing with tears, and has very low efficiency. The intraocular injection has high efficiency but is accompanied by pain. The insertion of a drug by surgery has various side effects. Therefore, a drug delivery system is needed to minimize side effects.

Meanwhile, according to the development of a light emitting diode (LED) and enhancement of an LED structure, it is possible to develop an LED having high efficiency in various wavelength bands. In addition, there are various application methods of the LED, such as a flexible LED made by transfer to a flexible material in addition to an LED using a transparent electrode.

### DISCLOSURE

### Technical Problem

The present invention is directed to providing the development of a smart remotely controlled contact lens for diagnosing and treating a disease using a micro light emitting diode (LED) or organic light emitting diode (OLED).

### Technical Solution

The present invention provides a smart remotely controlled contact lens for diagnosing and treating a disease, which includes a micro LED or OLED.

### Advantageous Effects

In the present invention, the diagnosis and treatment of a disease can be possible using a micro light emitting diode (LED) or organic light emitting diode (OLED) in a contact lens.

In addition, various diseases can be treated by controlling drug release from a drug reservoir in the contact lens with a signal in response to a light wavelength using a photodetector. A small drug reservoir that can be inserted into an eye can be electrically controlled. Accordingly, since a drug is released when desired, the photodetector can be applied to treat various diseases. The photodetector can also detect a therapeutic effect in real time due to light reflected from target cells which have been treated, and thus can easily and rapidly confirm the progression of a patient's disease.

In addition, by using a therapeutic method for consistently applying light by the integration of a short-wavelength LED or OLED in a contact lens, a disease can be easily treated while sleeping or when the contact lens is worn, it is possible to solve a shortcoming of surrounding cells being damaged due to an LED light source of a conventional therapeutic device.

Whiles a conventional contact lens was driven by wirelessly receiving power from the outside, the present invention can provide an operable smart contact lens using a battery, without external power supply.

In addition, in the present invention, power consumption can be significantly reduced by controlling drug release by analyzing data detected in a sensor in the lens without wireless data transmission.

### DESCRIPTION OF DRAWINGS

FIG. 1 is a schematic view of a smart contact lens according to an exemplary embodiment of the present invention.

FIGS. 2 to 4 are schematic views of an exemplary smart contact lens including a micro light emitting diode (LED):

Specifically, FIG. 2 is a schematic view of a smart contact lens for diagnosing an eye disease using a micro LED light source, FIG. 3 is a schematic view of a smart contact lens for treating retinitis pigmentosa using a micro LED light source, and FIG. 4 is a schematic view of a smart contact lens for treating macular degeneration using a micro LED light source and a drug delivery system.

FIG. 5 is a set of schematic views of a drug delivery system in a smart contact lens.

FIG. 6 is a schematic view of a system for real-time monitoring of cells using a smart lens by injecting therapeutic cells into the aqueous humor.

FIGS. 7a and 7b are graphs showing cell viability according to glucose concentration.

FIG. 8 is a thermal image profile showing the result obtained immediately after a lens is operated, and FIG. 9 is a thermal image profile showing the result obtained after a micro LED is continuously operated for 10 minutes at 1.6V.

FIG. 10 is a graph of the current intensity of a photodetector according to glucose concentration at a wavelength of 1,050 nm.

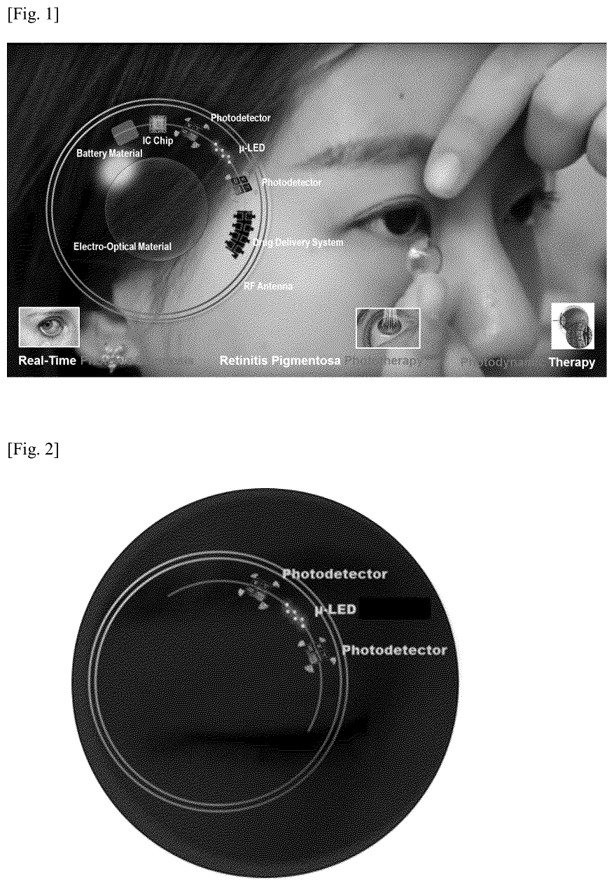

### MODES OF THE INVENTION

The present invention relates to a smart remotely controlled contact lens for diagnosing and treating a disease, which includes a micro light emitting diode (LED) or organic light emitting diode (OLED).

Hereinafter, the smart remotely controlled contact lens will be described in detail.

In the present invention, the type of a disease may be, but is not particularly limited to, a systemic disease or an eye disease (ophthalmic disease). The systemic disease may be diabetes or depression, and the eye disease may be increased intraocular pressure, glaucoma, uveitis, retinal vein occlusion, macular degeneration. diabetic retinopathy, various types of macular edema, postoperative inflammation, an inflammatory disease of the eyelid and ocular conjunctiva such as allergic conjunctivitis, an inflammatory disease of the cornea or anterior eye, eye injections, dry eye, blepharitis, retinal detachment, depression, dry eye syndrome, retinitis pigmentosa, Meibomian gland dysfunction, superficial punctate keratitis, herpes zoster keratitis, iritis, ciliary inflammation, selective infectious conjunctivitis, corneal injury from a chemical, radiation or thermal burn, invasion of foreign matter, or an allergic disease.

The smart remotely controlled contact lens according to the present invention includes a micro LED or OLED.

The micro LED or OLED may be a product used in the art, or may be directly manufactured. Generally, the micro LED or OLED may have an epitaxial layer on a substrate. The substrate may be silicon carbide (SiC), gallium arsenide (GaAs) or a silicon wafer (Si wafer).

The micro LED or OLED may play various roles in the smart contact lens, and particularly, may play a diagnostic or therapeutic role.

The micro LED or OLED according to the present invention may be used in diagnosis, and the micro LED or OLED may diagnose a disease or determine whether a disease is treated by applying light to a disease marker.

When used for diagnosis, the smart contact lens may include a photodetector in addition to the micro LED or OLED. For example, the micro LED may apply light to a disease marker, the photodetector may detect and analyze reflected light, thereby determining whether a disease, that is, a disorder, is diagnosed or treated.

The micro LED may be a near-infrared (NIR) LED. When the NIR-LED is used in the present invention, as a photodetector, an IR detector may be used. The IR detector is one type of photodetector, which easily detects IR light with a long wavelength.

Measurement of oxygen saturation in the eye may allow early diagnosis of diseases such as retinal hypoxia, glaucoma and perfusion, which may be distinguished using the difference in absorbance according to oxygen saturation of hemoglobin. Particularly, since absorbance differs at wavelengths of 660 nm and 940 nm, eye diseases may be diagnosed early by measuring oxygen saturation at these two wavelengths.

For example, diabetes may be diagnosed by measuring a sugar level using the NIR-LED, or an eye disease may be diagnosed by measuring oxygen saturation based on oximetry.

In addition, in the present invention, a concentration of glucose in blood, not in body fluids, may be analyzed in real time by measuring glycated hemoglobin levels in capillaries of eyelids in contact with each other when eyes are closed. When the LED light source is applied to the blood vessels in the retina or eyelid, the extent of LED light absorption varies depending on the concentration of a disease marker in the blood vessels. The photodetector may measure an amount of light that is reflected and then returns to assess an amount of a disease marker, thereby diagnosing a disease. That is, light at wavelengths of 660 and 940 nm is applied using the micro LED, and oxygen saturation may be measured using the photodetector by detecting the difference in absorbance according to oxygen saturation in hemoglobin in capillaries of the eyelid.

As another example, the photodetector may measure the difference in the intensity of light between the wavelengths of glucose or glycated hemoglobin in blood, and oxygen or oxyhemoglobin, and diagnose a disease by analyzing a blood glucose concentration, oxygen partial pressure and oxygen saturation. Diabetes may be diagnosed by analyzing a glucose concentration in the blood vessel, and macular degeneration, glaucoma and cataracts, which are directly associated with an ocular oxygen level, may be diagnosed. In addition, in the present invention, a raw data is transmitted wirelessly, and an analysis result of the photodetector may be transmitted to the outside using a wireless transmission system that can immediately confirm the diagnostic result.

The micro LED and photodetector of the present invention may be integrated on a flexible substrate through a transfer process by adding a sacrificial layer in the epitaxial growth of each layer. In addition, a diagnostic system for measuring oxygen saturation based on oximetry may be formed using a flip chip bonding process. The micro LED or OLED and the photodetector may adjust a wavelength depending on the control of a composition ratio and the selection of a material during the growth process, and ultimately, it is possible to diagnose oxygen saturation and accompanying eye diseases through irradiation and detection at wavelengths of 660 nm and 940 nm.

In addition, the micro LED or OLED according to the present invention may be used to express the presence or absence of a disease marker or a concentration, which is detected by a sensor. In this case, the smart contact lens may include a sensor and a photodetector, in addition to the micro LED or OLED. Accordingly, the sensor detects a disease marker, the micro LED or OLED may express the presence or absence of the disease marker or the concentration of the disease marker by light, and the photodetector may detect light of the LED or OLED and analyze it, thereby diagnosing a disease or determining whether the disease is treated.

In the prior art, the diagnosis results of the sensor were transmitted to the outside using wireless communication, but this method consumed a lot of energy. In the present invention, the photodetector may analyze light from the LED or OLED, or may externally determine the presence or absence of a disease by changing a color depending on a level of the disease marker and sending the diagnosis result to the outside the contact lens. That is, a sensor alarm function may be performed.

The sensor may be any sensor that can detect a disease marker in the eye without particular limitation, for example, a glucose sensor or a pressure sensor. In addition, the disease marker may be one or more selected from the group consisting of nitrogen monoxide, a vascular epidermal growth factor (VEGF), an epidermal growth factor (EGF), a monosaccharide containing glucose, a disaccharide containing glucose, a water content, flavin adenine dinucleotide (FAD), bovine serum albumin (BSA), hydrogen peroxide, oxygen, ascorbic acid, a lysozyme, iron, lactoferrin, a phospholipid, osmotic pressure and intraocular pressure.

In one embodiment, when a glucose sensor is used, the glucose sensor diagnoses a glucose concentration, the content is determined in an IC chip, and the micro LED may express the glucose concentration as a color. The photodetector may diagnose a disease or determine whether the disease is treated by analyzing the wavelength of the LED color.

The micro LED may be a blue light LED or NIR-LED.

In another embodiment, the photodetector may apply light to a disease site by the micro LED for treatment, and then detect the reflected light, thereby confirming a therapeutic effect in real time.

The smart contact lens according to the present invention may further include a drug reservoir. The drug reservoir may be connected with the photodetector, and opened in diagnosis of a disease by the photodetector. Specifically, drug release from the drug reservoir installed in the lens may be controlled by various signals according to a wavelength of external light using the photodetector.

In the present invention, the drug reservoir may be formed in a drug well, where the inner surface of the smart contact lens in contact with the eye ball may be drawn toward the outside, and which may be sealed by an electrode pattern.

The drug reservoir may contain a drug; or a drug carrier capable of releasing a drug and a drug release control material.

In the present invention, as the drug reservoir. a drug reservoir disclosed in Korean Unexamined Patent Application No. 10-2016-0127322 may be used.

In addition, the drug reservoir may be manufactured by the following preparation method for use. The preparation method is simplified and production costs may be reduced by the following method.

The preparation method may include:
(a) preparing a drug storage mold;
(b) loading a drug in the mold;
(c) attaching an electrode-deposited hydrophilic polymer film to the mold; and
(d) performing passivation.

In step (a), the mold may be a polydimethylsiloxane (PDMS) mold, and may be prepared using a mold frame. A size of the mold may be suitably adjusted according to a content of the stored drug and a size of the lens, and the mold may have a plurality of drug storage wells.

In step (c), electrodes are deposited on a hydrophilic polymer film, and then attached to the mold. Here, the type of hydrophilic polymer is not particularly limited as long as the hydrophilic polymer is dissolved in water, and for example, polyvinyl alcohol (PVA) may be used. The electrodes, such as a positive electrode and a negative electrode, may be prepared by patterning with Ti and Au, respectively.

In step (d), for insulation and water-proofing, the mold is passivated. The passivation may be performed using SiO₂ passivation according to a method known in the art.

In addition, the micro LED or OLED according to the present invention may be used in treatment of a disease, in addition to diagnosis of the above-mentioned disease.

The micro LED or OLED may treat a disease by applying light to a disease site.

In the present invention, as a light therapy system for treating a disease is introduced into the smart contact lens, and a biocompatible nanomaterial for manufacturing an LED or OLED mediating multi-wavelength light transmission to the body is developed, side effects of the existing treatment techniques may be overcome by avoiding an invasive method by surgery and precisely controlling nerve cells in a desired region. Specifically, since non-invasive light therapy using multi-wavelength light transmission into the body enables treatment at a single cell level, this method can compensate for the risk of random expression of side effects of drug treatment which has been conventionally performed for disease treatment.

In addition, compared with a DBS therapy in which an invasive probe is implanted or a current light therapy system in which a light fiber is surgically implanted into a neurological disorder target site to deliver visible rays into the body, which has been suggested as various alternative techniques of drug treatment, the technology of the present invention may be highly applicable for clinical applications and various applications, may significantly reduce the probability of bleeding and infection, and may be effectively and selectively applied to disease treatment using light. Accordingly, source technology of the next-generation system for treating a neurological disease may be ensured.

In the present invention, a disease may be treated by applying light from the micro LED or OLED in the smart contact lens to the retina, and such a disease may be a systemic disease or an eye disease. The systemic disease may be diabetes or depression, and the eye disease may be increased intraocular pressure, glaucoma. uveitis, retinal vein occlusion, macular degeneration, diabetic retinopathy, various types of macular edema, postoperative inflammation, an inflammatory disease of the eyelid and ocular conjunctiva such as allergic conjunctivitis, an inflammatory disease of the cornea or anterior eye, eye injections, dry eye, blepharitis, retinal detachment, depression, dry eye syndrome, retinitis pigmentosa, Meibomian gland dysfunction, superficial punctate keratitis, herpes zoster keratitis, iritis, ciliary inflammation, selective infectious conjunctivitis, a corneal injury from a chemical, radiation or thermal burn, invasion of foreign matter, or an allergic disease.

The smart contact lens may include an LED or OLED emitting light with a specific wavelength for treating each disease, and the LED may be a blue light LED or NIR-LED.

In one embodiment, the micro LED or OLED may be used for treatment of age-related macular degeneration (AMD). One of the factors causing AMD is the lipofuscin fluorophore A2E. The lipofuscin fluorophore A2E accumulated in retinal pigmented epithelium cells is the cause of aging and a retinal disorder. Such lipofuscin fluorophore A2E is damaged by blue light (420 nm). Therefore, when a blue LED is installed in the smart contact lens, it is expected to be effective in AMD treatment.

To this end, in the present invention, using a Merck blue (poly(9,9-di-n-octylfluorenyl-2,7-diyl); PFO) material exhibiting blue light in a wavelength range from 420 nm to 600 nm, applicable to the macula, an OLED for AMD treatment may be manufactured. Alternatively, a NIR-LED may be used.

In one embodiment, a system for light therapy for an eye disease may be provided by integrating a blue light LED in the smart contact lens. Currently, research for overcoming seasonal depression or biorhythms using blue light is widely progressing, and when blue light is applied using the smart contact lens, since light transmission efficiency to the eyes is high, and blue light can be transmitted even when a patient closes his eyes, patient convenience may be improved, and therapeutic efficiency may be dramatically enhanced.

In another embodiment, retinitis pigmentosa may be treated by repeatedly stimulating the optic nerve of the retina at regular intervals using a blue light LED to recover the optic nerve.

In addition, the micro LED or OLED of the present invention may treat a disease in combination with a drug reservoir.

In one embodiment, by combining the micro LED or OLED with the drug reservoir, macular degeneration may be treated. In this case, a photosensitizer producing active oxygen in response to light may be used, when the photosensitizer is released from a drug delivery system and transferred to a retinal blood vessel according to an instruction from the photodetector, active oxygen is generated using light of the micro LED or OLED and thus can be used in treatment of macular degeneration. The efficiency of generating active oxygen may be increased by the photosensitizer, which, therefore, can be used in treatment of an angiogenic disease caused in the macula. As such a photosensitizer, visudyne clinically used or a block phosphorus known as a two-dimensional new material may be used. Particularly, in the present invention, an on-off system may be manufactured to generate active oxygen in a small amount so that surrounding normal blood vessels may not be damaged while the smart contact lens is worn.

The combined treatment of the micro LED or OLED with the drug reservoir may be applied to various diseases such as diabetic retinopathy and choroidal neovascularization as well as macular degeneration.

The smart contact lens according to the present invention may further include a thin film-type battery which has a thickness of 300 μm or less or 50 μm or less, and has flexibility. The lower limit of the thickness of the thin film-type battery may be 1 μm.

Wireless driving of the smart contact lens is possible using the thin film-type battery. A conventional smart contact lens receives energy by wireless power transmission using a coil to operate a system. However, due to the low transmission efficiency of wireless power transmission, there is a problem in that energy has to be transmitted with a strong intensity using a coil from the outside. Accordingly, the application of the smart contact lens may not only be highly limited, but also cause inconvenience in use. In addition, for intraocular pressure monitoring using a contact lens, a Sensimed Triggerfish contact lens sensor was conventionally used, which may not only limit a user's vision using an opaque metal antenna and a strain sensor, installed in the lens, but also give repulsion. Since an external antenna for power supply has to be always attached to provide power and fixed to not shake, it is very difficult for many people to use such a sensor because there are limitations in use for many people due to considerable interference with everyday activities.

Therefore, in the present invention, the above-mentioned problem may be solved by installing a thin film-type battery in the smart contact lens. That is, in the present invention, an operable system may be implemented without providing power from the outside to operate the smart contact lens system using a micro thin film battery.

The battery may further simplify the smart contact lens by storing electric power in the battery from various energy sources such as light energy, piezoelectric energy and/or thermal energy. The battery may provide electric power to the elements constituting the contact lens. In addition, there is no battery damage despite repeated bending or deformation, and when the battery is applied to the lens, it is sealed and may ensure intraocular stability.

The battery of the present invention may have a thickness of 300 μm or less or 50 μm or less, and have flexibility. Since the battery is installed in the lens, there is a limit in size, and thus a battery having a thickness of 300 μm or less is preferably used for convenience in use.

Specifically, the battery of the present invention may be a thin film-type flexible lithium ion battery having a thickness of 300 μm or less. The lithium ion thin film-type battery does not need an external antenna for power supply, may eliminate a user's hassle of wearing and inconvenience in life, and may eliminate the limitation in vision and repulsion by removing the antenna in the lens.

In one embodiment, the battery may be charged by a coil. Particularly, due to the insertion of a transparent coil into the lens, wireless charging is possible when the lens is not used.

The thin film-type battery of the present invention may be a product used in the art, or may be directly manufactured.

In one embodiment, the battery may be formed of a polymer/silver nanoparticle composite material and a block copolymer fiber/active material composite material.

In the present invention, to provide power to the micro LED or OLED, the micro LED may be connected with the above-described battery, and for intraocular stability, the micro LED and the battery may be passivated with a PDMS polymer.

In addition, in the present invention, a wireless electrical system for sending and receiving data wirelessly may be further included.

In the smart remotely controlled contact lens according to the present invention, after being integrated on a substrate, components such as a micro LED or OLED, an ASIC chip, a battery and a drug reservoir may be included in the contact lens. Here, the substrate may be poly (ethylene terephthalate) (PET), poly (propylene) (PP), polyamide (PI), poly (ethylene naphthalate) (PEN), poly (ether sulfones) (PES) or polycarbonate (PC).

The smart remotely controlled contact lens according to the present invention may be based on a polymer of poly (2-hydroxyethylmethacrylate) (PHEMA), poly (methyl methacrylate) (PMMA), poly (lactide-co-glycolide) (PLGA), polyvinylpyrrolidone (PVP), polyvinyl alcohol (PVA) or silicone hydrogel.

In addition, a super thin contact lens may be manufactured to have a thickness of 100 μm or less using radial polymerization by molding a PET-based blue LED in a poly (2-hydroxyethylmethacrylate) (PHEMA)-based.

The smart contact lens according to the present invention may further include an active element that can control the wavefront of light in the lens.

In the present invention, images with various degrees of freedom may be acquired or implemented by applying a suitable appropriate phase delay pattern to the active element. For example, by changing the focal distance of the contact lens by recognizing a user's action (e.g., when reading a book with his head down), the user can see a nearby place easily. In addition, by controlling various phase delay values per section of the active element of the contact lens, improved light transmission/control to the retina may be realized. That is, a very small optical focus may be achieved at a sub-micrometer level at various positions of the retina by applying an adaptive optics technique.

In the present invention, irradiation of a part required for treatment can be continuously performed by integrating a short-wavelength LED on the smart contact lens and integrating an active element that enables the optical design and control of the focal distance. Therefore, a method which was only possible to be performed at a determined time in a dark place, which is conventionally limited space-time may allow easy treatment in sleep or when the contact lens is worn, and may also resolve a shortcoming in that a laser can easily damage surrounding cells. Such an active element may use a liquid crystal and a material enabling the control of a refractive index.

In addition, the present invention may further include an optical sensor or an image sensor.

The color of cells is changed according to a condition, and particularly. when there is a disease, when a blood vessel is generated in the cells, there are more and more red blood vessels and the cells become reddish. In the present invention, an optical sensor detecting such light may be additionally used, thereby enhancing disease diagnosis efficiency. In addition, monitoring may be performed by determining the color of cells using an image sensor. In addition, in the present invention, cells may be monitored in real time using the smart contact lens after the therapeutic cells are injected into the aqueous humor (FIG. 6).

In the present invention, the smart contact lens may be controlled with low power by a communication method using a light signal to control the smart contact lens.

Specifically, the operation of a system may be controlled by transmitting data to the smart contact lens using an external light signal to control the smart contact lens.

In addition, in the present invention, all of the wiring, pad and coil parts are manufactured of a transparent material so that a patient has no limitation in vision and does not feel awkward when seen from the outside.

In addition, the present invention may provide an energy harvesting system which harvests electrical energy converted from light energy using a solar light generating element and uses the electrical energy as an energy source to replenish energy required for driving the system.

### EXAMPLES

### Preparation Example 1. Manufacture of μLED for Smart Contact Lens

P-GaN/multi quantum well (InGaN/GaN)/N—GaN/buffer layer/GaN (blue μLED) and (Al_{0.45}Ga_{0.55}As:C)/(In_{0.5}Al_{0.5}P:Zn)/multi quantum well (Al_{0.25}Ga_{0.25}In_{0.5}P/In_{0.50}Ga_{0.44}P/Al_{0.25}Ga_{0.25}In_{0.5}P)/(In_{0.5}Al_{0.5}P:Si)/(Al_{0.45}Ga_{0.55}As:Si)/n-GaAs:Si (near infrared μLED), which constitute an epitaxial layer, was manufactured on a substrate.

The shape of the μLED was patterned on the manufactured substrate through photolithography. Electrodes were connected to the patterned μLED by the application of plasma etching and metal wiring techniques. After palladium was deposited on the completed element, palladium-indium was connected to a silicon substrate coated with palladium and indium. A sapphire substrate was removed using a laser lift-off (LLO) technique, the connection between the substrate and the μLED became loose through under-cut etching, and then the μLED was electrically connected with a circuit in the contact lens through transfer printing.

### Preparation Example 2. Manufacture of Drug Reservoir

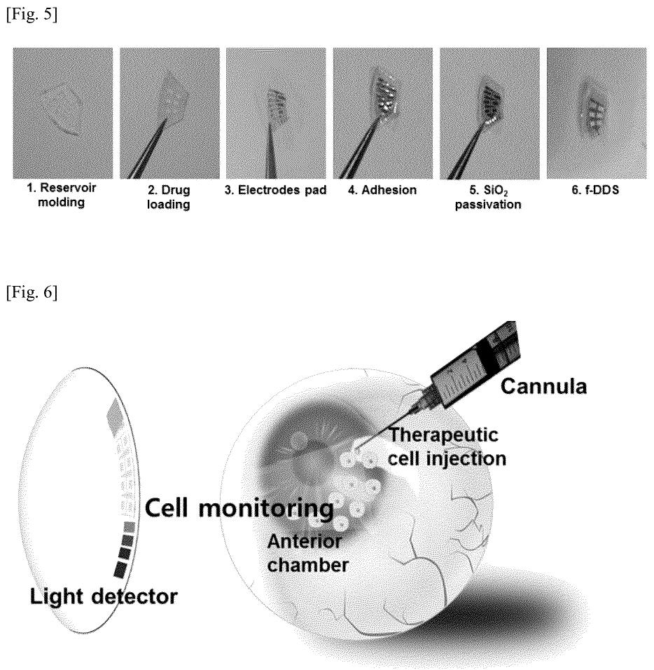

A drug reservoir was manufactured by the method shown in FIG. 5.

First, a PDMS mold was manufactured using a mold frame, and then a drug was loaded in the reservoir.

After electrodes (a negative electrode and a positive electrode formed of Ti and Au, respectively) were deposited on a polyvinyl alcohol (PVA) film, an electrode-deposited PVA film was attached to the PDMS mold containing the drug. Subsequently, for insulation and water-proofing, SiO₂ passivation was performed, thereby manufacturing the drug reservoir.

### Preparation Example 3. Process of Integrating Elements on Polymer Substrate

To integrate an ASIC chip, a photodetector. the μLED manufactured in Preparation Example 1 and a battery. 100-nm gold was formed on a 30-μm or less PET substrate by thermal deposition, or for post processing, a metal film was formed with a structure of titanium (Ti: 10 nm)/aluminum (Al: 500 nm)/titanium (10 nm)/gold (Au: 50 nm).

Afterward, patterning was performed in a required shape through photolithography. The patterning process was performed according to the structure of the metal film through a lift off method, wet etching or dry etching using a negative or positive photoresist.

A gold bump was formed on the patterned polymer substrate to bond the metal film with each element. Here, the bump had a diameter of 15 to 50 μm and a height of 10 to 20 μm.

The Gold bump-formed substrate was bonded with the ASIC chip, the photodetector, the μLED and the drug reservoir using a flip chip bonding technique.

### Preparation Example 4. Manufacture of Contact Lens

A contact lens was manufactured using a silicone-containing material.

First, 1 mL of methacryloxypropyl-tris (trimethylsiloxy) silane was mixed with 0.62 mL of N,N-dimethyl acrylamide (DMA). 1 mL of methacryloxypropyl (MC)-PDMS macromere, 0.3 mL of methyl acrylic acid (MAA), 0.1 mL of ethanol, and 0.2 mL of N-vinylpyrrolidone (NVP) for 15 minutes in a nitrogen environment. In addition, 12 μg of TPO as an ultraviolet (UV) initiator was added to the resulting mixture for 5 minutes, thereby preparing “solution 1.”

Following radical polymerization in a specially-manufactured polypropylene (PP) mold using 0.2 mL of the prepared Solution 1, the polymer surface was made hydrophilic using ozone plasma, and then stored in a PBS solution.

Afterward, a lens was manufactured by radical polymerization in the PP mold after the substrate on which the ASIC chip, the photodetector, the micro LED, and the battery (the micro battery manufactured by Cymbet) were integrated, which was manufactured in Preparation Example 3, was added into the mold.

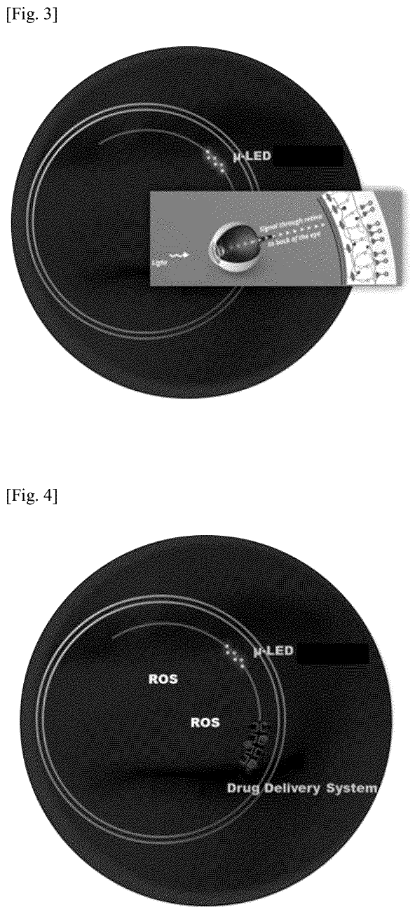

The components included in the smart contact lens may vary depending on the use of the micro LED. As shown in FIGS. 2 to 4, the configuration of the contact lens may vary depending on its use, and a contact lens including all the components as shown in FIG. I may be manufactured.

### Experimental Example 1. Confirmation of Therapeutic Effect of NIR Light in Cells

The therapeutic effect of NIR light in cells was confirmed using ARPE-19 cells.

The ARPE-19 cells were incubated in a normal glucose concentration environment (glucose concentration: 5 mM) and a high glucose concentration environment (glucose concentration: 30 mM) at 37° C. under a 5% CO₂ condition.

Light was irradiated using a NIR-LED twice daily for 5 days of incubation.

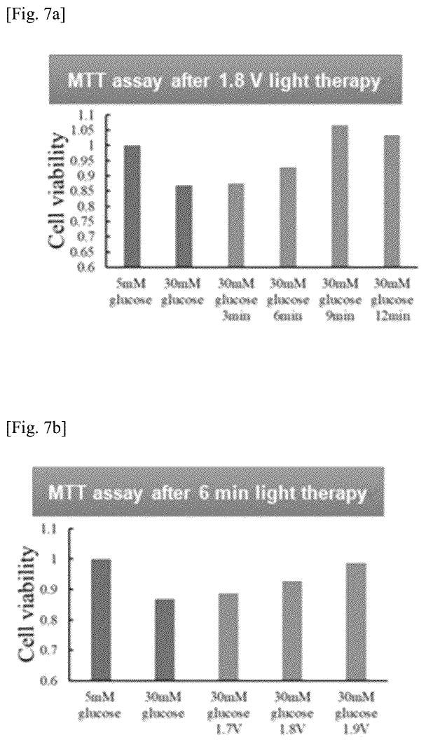

In the present invention, FIGS. 7a and 7b are graphs showing cell viability according to glucose concentration.

As shown in FIG. 7b, compared with the normal environment, in a high glucose concentration environment, it was confirmed that cell viability decreases. However, when light was irradiated using a NIR-LED at a voltage of 1.8V, it was confirmed that, in the high glucose concentration environment, compared with the normal environment, cell viability was similar (FIG. 7a).

Meanwhile, as the voltage applied to the LED increases, it can be confirmed that cell viability tended to increase.

### Experimental Example 2. Confirmation of Therapeutic Effect of NIR Light in Animal

An animal experiment was performed using rats.

After a lens was manufactured to fit the curvature of a rat's eye, an NIR-LED was attached to the lens to confirm a therapeutic effect.

Treatment was performed for 5 days, and the experiment was performed on the rats divided into groups. while changing the intensity of the LED.

### Experimental Example 3. Confirmation of Heat Generation in LED Contact Lens

A heat generation experiment was performed using rats.

After a contact lens was manufactured to fit the curvature of a rat's eye, an NIR-LED was attached to the lens to confirm heat generated in the LED using a thermal imaging camera.

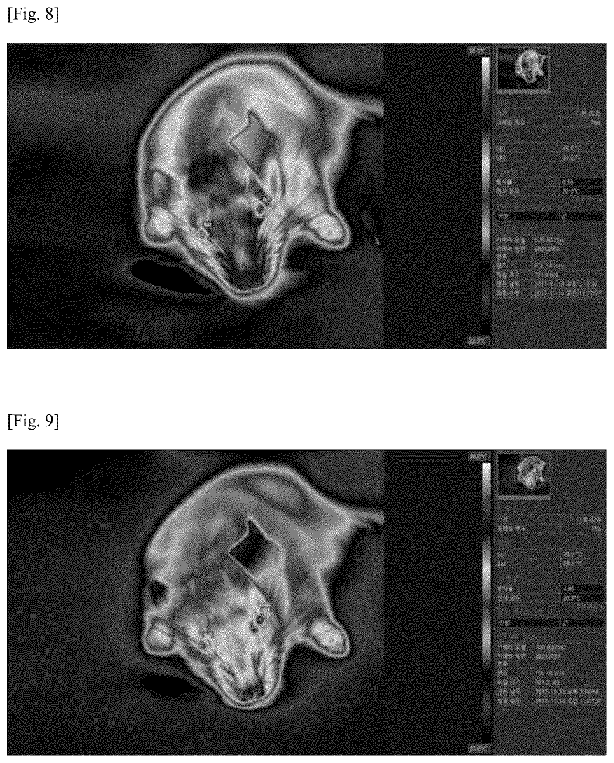

In the present invention, FIG. 8 is a thermal image profile showing the result obtained immediately after a lens is operated, and FIG. 9 is a thermal image profile showing the result obtained after a micro LED is continuously operated for 10 minutes at 1.6V. In addition, in FIGS. 8 and 9, there was no contact lens worn on the left eye, and there was a contact lens worn on the right eye.

As shown in FIGS. 8 and 9, it was confirmed that the temperature difference between both eyes of the rat was 1° C. or less.

Experimental Example 4. Diagnosis of glucose concentration using NIR light.

Blood samples having different glucose concentrations (0.6, 1.1. 1.6 and 2.1 mg/ml) were prepared.

A blood sample was placed in a cuvette, and then an NIR-LED (730, 850, 950, 1050, 1450 or 1550 nm) was installed on one side, and a photodetector was installed on the other side.

The blood sample was replaced and a current intensity of the photodetector was measured according to glucose concentration.

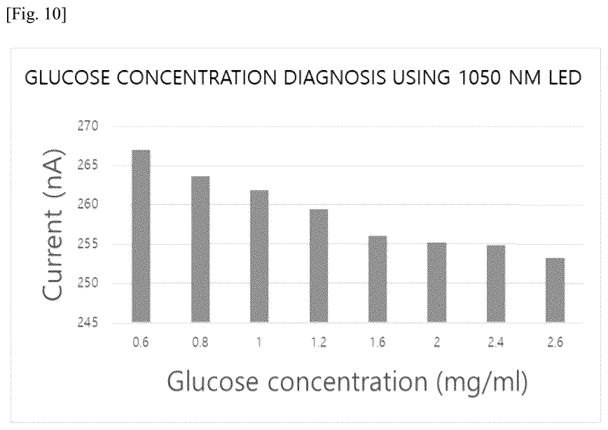

In the present invention, FIG. 10 is a graph of the current intensity (nA) of a photodetector according to glucose concentration at a wavelength of 1,050 nm.

As shown in FIG. 10, as a result of using an LED with a wavelength of 1,050 nm, it can be seen that, as the concentration increases, the value of a current flowing through the photodetector is reduced.

### INDUSTRIAL APPLICABILITY

In the present invention. the diagnosis and treatment of a disease are possible using a micro light emitting diode (LED) or organic light emitting diode (OLED) in a contact lens.

In addition, various diseases can be treated by controlling drug release from a drug reservoir in the contact lens with a signal according to a light wavelength using a photodetector. A small drug reservoir that can be inserted into the eye can be electrically controlled. Therefore, since drug release when desired is possible, the drug reservoir can be applied for treatment of various diseases. Since the photodetector can detect a therapeutic effect in real time by light reflected from treated target cells, the progression of a patient's disease can be easily and quickly confirmed.

In addition, by a treatment method of integrating a short-wavelength LED or OLED in the contact lens and continuously irradiating light, a disease can be easily treated while sleeping or when the contact lens is worn, a shortcoming of surrounding cells being easily damaged by an LED light source of a conventional treatment device may be resolved.

1-16 (canceled)

17. A method for diagnosing and treating a disease using a smart remotely controlled contact lens which comprises a micro light emitting diode (μLED) or an organic light emitting diode (OLED), and a photodetector; or comprises the micro LED or OLED, the photodetector, and a sensor,
wherein the disease is diabetes, glaucoma, macular degeneration, retinitis pigmentosa, cataracts, increased intraocular pressure, diabetic retinopathy, dry eye syndrome or Meibomian gland dysfunction,
wherein the micro LED or OLED applies light to a disease marker, and the photodetector detects and analyzes reflected light to diagnose the disease or determine whether the disease is treated, or
the sensor detects a marker, the micro LED or OLED expresses the presence or absence of the marker or a concentration of the marker by light, and the photodetector detects and analyzes light of the LED or OLED, and
wherein the LED or OLED detects disease markers of capillaries of eyelids in contact with each other when the eyes are closed, and wherein the disease marker is glucose or glycated hemoglobin, or oxyhemoglobin.

18. The method of claim 17, wherein the photodetector analyzes the glucose concentration, oxygen partial pressure and oxygen saturation by measuring the difference in light intensity according to wavelength.

19. The method of claim 17, wherein the sensor detects one or more selected from the group consisting of nitrogen monoxide, a vascular epidermal growth factor (VEGF), an epidermal growth factor (EGF), glucose-containing monosaccharides, lactose-containing disaccharides, a water content, flavin adenine dinucleotide (FAD), bovine serum albumin (BSA), hydrogen peroxide, oxygen, ascorbic acid, a lysozyme, iron, lactoferrin, a phospholipid, osmotic pressure, and intraocular pressure.

20. The method of claim 17, wherein the micro LED is a blue light LED or a near-infrared (NIR)-LED.

21. The method of claim 20, wherein a drug reservoir is opened when a disease is diagnosed by the photodetector.

22. The method of claim 17, further comprising a thin film-type battery, which has a thickness of 300 μm or less and flexibility.

23. The method of claim 22, wherein the thin film-type battery is a lithium ion thin-film-type battery.

24. The method of claim 17, wherein the micro LED or OLED is integrated on the substrate, and the substrate is poly (ethylene terephthalate) (PET), poly (propylene) (PP), polyamide (PI), poly (ethylene naphthalate) (PEN), poly (ether sulfones) (PES) or polycarbonate (PC).

25. The method of claim 17, further comprising a wireless electrical system for sending and receiving data wirelessly.

26. The method of claim 17, further comprising an active element.

27. The method of claim 17, further comprising an optical sensor or an image sensor.
